(19) United States
(12) Patent Application Publication
(10) Pub. No.: US 2006/0030620 A1
(43) Pub. Date: Feb. 9, 2006

(54) Method for treating and/or preventing ischemia/reperfusion injury

(76) Inventors: Chia-Yu Chang, Tainan City (TW); Shui-To Wang, Tainan City (TW); Sheng-Yuan Huang, Jiading Township (TW); Jeng-Chai Huang, Tainan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/910,295

(22) Filed: Aug. 4, 2004

(51) Int. Cl. A61K 31/375

(52) U.S. Cl. 514/474

(57) ABSTRACT

Magnesium ascorbate, a previous Romanian cardio protective drug, is provided to alleviate ischemia/reperfusion injury. In particular, the present invention relates to a pharmaceutical composition of magnesium ascorbate for treating and/or preventing liver ischemia/reperfusion injure in mammals.

### BACKGROUND OF THE INVENTION

1. Field of the Invention

The present invention relates to a method for treating and/or preventing ischemia/reperfusion injury in mammal, particularly to a method for attenuating ischemia/reperfusion injury by administering a mammal with a pharmaceutical composition comprising magnesium ascorbate (MA).

2. Description of Related Prior Arts

As lack of blood (or oxygen-rich blood), ischemia may cause ionic disequilibrium or constant injury of organs or tissues. When ischemia lasts over 10 minutes, reperfusion could cause further injury. Oxidative stress is known as one of the major injuries that occurs mostly when oxygen is reintroduced to ischemia tissue to generate free radicals through conventional Fenton reaction.

For example, liver ischemia/reperfusion (I/R) injury involving pathogenic shock could occur after liver transplantation and hepatic surgery for trauma or cancer. Report showed elevated lipid peroxide in the liver tissue following I/R, which suggested the involvement of oxidative stress in I/R injury (Kudo Y, et al., “Investigation of the renal injury caused by liver ischemia/reperfusion injury in rats”, Arch Toxicol, 1993; 67:502-509).

Many antioxidants are proposed to alleviate injuries from reperfusion-generated free radicals. For example, ascorbic acid and tocopherols are used for opposing oxidative stress by reducing the reactive oxidants. Ascorbic acid can preserve cardiac tissue and reduce oxidative indices after I/R. Paradoxically, ascorbic acid is also associated with pro-oxidant effects, which is known for its ability to reduce metals into the form that reacts with oxygen to produce lipid peroxidation initiators. Both the effects and mechanisms of ascorbic acid on post-ischemia tissue injury are known in the art.

On the other hand, magnesium sulfate is used to rescue the neurons from the rat's heart attacked by I/R syndrome. Compared with its lower dosage (8 mMol of MgSO₄), there is no significant increase in serum magnesium concentration, but the neuron-protective effect of parenteral magnesium sulfate is obvious. Such protective effect has several potential mechanisms, including blockade of non-competitive N-methy-D-aspartate receptor, and enhancement of regional cerebral blood flow to areas of focal ischemia.

Magnesium ascorbate (MA; trade mark “Magnorbin”, Merck (Darmstadt, German)) is a known pharmaceutical and indicates as cardiac protector for reducing lipid peroxidation when anthracycline antibiotic is administered for a long period. This MA is also known to attenuate large dose of beta adrenergics; isoproterenol induced myocardial disease by return to the normal levels of ATP and Mg⁺², and decrease in uptake of Ca⁴⁵Cl₂ by myocardium. In other words, MA possesses both the ascorbate and magnesium properties.

Therefore, this invention provides a method for attenuating liver ischemia/reperfusion injury by administering a mammal with a pharmaceutical composition comprising magnesium ascorbate.

### SUMMARY OF THE INVENTION

It is an object of the invention to provide a method for treatment and/or preventing ischemia/reperfusion injury.

In this method, a therapeutically effective amount of magnesium ascorbate is administered to a mammal which may suffer to ischemia/reperfusion injure.

The present invention also provides a pharmaceutical composition for treatment and/or preventing ischemia/reperfusion injure, which primarily comprises magnesium ascorbate as an active ingredient and a pharmaceutically acceptable carrier.

Among all ischemia/reperfusion injuries, that occurring in liver may cause a lethal result, and thus the present invention are exemplified by liver ischemia/reperfusion injure.

### BRIEF DESCRIPTION OF THE DRAWINGS

FIG. 1 shows the plasma aspartate transaminase (AST) levels of rats determined during the I/R process.

FIG. 2 shows the plasma alanine transaminase (ALT) levels of rats determined during the I/R process.

FIG. 3 shows the bile flow of rats measured during the I/R process.

FIG. 4 shows the alkaline phosphatase (ALP) levels determined during the I/R process.

FIG. 5 shows the compared difference of malondialdehyde (MDA) content in the livers with or without 70 min of ischemia and reperfusion treatment and the effective of MA on I/R injury.

FIG. 6 shows the survival rate during the I/R process.

FIGS. 7A, 7B and 7C show the hepatic tissue examined by H&E stain after 70 minutes of ischemia and subsequently 120 minutes of reperfusion.

FIG. 8 shows dry/wet ratios of group A, group B and control group.

### DETAILED DESCRIPTION OF THE INVENTION

Magnesium ascorbate (MA) is a buffered (non-acidic) form of vitamin C that will not contribute to gastric irritation in sensitive persons. MA is synthesized from a combination of ascorbic acid and magnesium to form magnesium ascorbate. In one embodiment of the invention, the MA is a buffered form of vitamin C, which containing approximately 87% ascorbic acid and 7.5% magnesium. Currently, MA is served as a dietary supplement in U.S.

As demonstrated in the Examples described below, the MA has demonstrated significantly improved therapeutic effects in treatment and/or prevention of liver ischemia/reperfusion (IR) injure in mammals.

As used herein, the term “treating”, “treatment”, “preventing”, or “prevention” when used with respect to the treatment of I/R injure refers to an attenuated hepatocytes necrosis and vessels endothelial damage observed after ischemia/reperfusion injuries, or a reduced MDA level generated during the I/R process, or an alleviated inflammatory effect (oxidative stress) generated during the I/R process, or an attenuated cholestasis occurred in the liver ischemia/reperfusion process.

### EXAMPLES

The following experiments are provided merely to further illustrate the protective effects of magnesium ascorbate (MA) on hepatobiliary function in liver ischemia/reperfusion injury. The scope of the invention shall not be limited by these experiments.

[Materials and methods]

Experimental Animals

60 male Spargue-Dawley rats, weighing 250 to 300 g each, were used in the experiments. The rats were cared in accordance with the guidelines from National Laboratory Animal Center in Taiwan and maintained under a 12-hour light/dark cycle in air-conditioned room level around 21° C. Animals were allowed free access to standard laboratory rodent chow diet and tap water. The rats were divided into 4 groups, each of which had 15 individual rats.

Hepatic Ischemia/Reperfusion Procedures

The rats were fasted for 24 hours before the experiment but allowed to drink water ad libitum. The animals were anesthetized with Urethane (500 mg/Kg, ip) (Sigma, St. Louise, Mo.), and placed in a supine position on a heating pad to maintain their thermal condition at 36˜37° C. with 200 IU/kg of heparin injected intravenously as anticoagulant. To induce hepatic ischemia, midline laparotomy was done and the blood supply to the left lobe of the liver was interrupted by placement of a bulldog clamp at the level of the hepatic artery and portal vein. The right lobe remained in perfusion to prevent intestinal congestion. After 70 min ischemia, the clamp was removed to restore the blood flow. The animals' bile juice was collected through a PE-10 polyethylene catheter (Intramedic, Clay Adams, Parsipany, N.J.) in the bile duct. The experimental rats' blood was collected from femoral artery through a PE-50 polyethylene catheter (Intramedic, Clay Adams, Parsipany, N.J.). A catheter with PE-50 tube to the femoral vein was connected for MA infusion purpose. Liver specimens were taken just before and after ischemia, and after 240-minute reoxygenation in different animals. The liver was then examined by the following assays. Since the above procedures were general and known by people skilled in this art, detailed operations were not described here.

Drug Administration and Sampling Procedure

Magnesium ascorbate (870 mg Vit-C and 75 mg Mg per 1.0 g; Now Foods, Bloomingdale, Ill.) was dissolved in saline (vehicle), filtered to sterilize by a 0.20 μm filter (Satorius AG, Gottingen, German), and administered intravenously with 100 mg/kg of body weight at 10 minutes prior to reperfusion. 4 different treatment groups of rats were as follows: (A) vehicle-treated ischemia group (or placebo group); (B) MA-treated ischemia group; (C) MA-treated control group (sham operation); and (D) vehicle-treated control group (sham operation). Those animals from either control group C or D subjected to the same surgical protocol but with neither ischemia nor reperfusion treatments referred as sham operation. Because there was no difference found in any of the parameters between control groups of MA-treated and vehicle-treated rats, the results of group C and D were pooled, and referred to as control group. Each treated group was followed up to 2 and 4 hours post treatment. 15 animals were used from each group for survival time assessment.

Analytical Procedures

The collected blood samples were centrifuged at 4000 g×5 min to separate the plasma, and the levels of aspartate transaminase (AST), alanine transaminase (ALT), and alkaline phosphatase (ALP) in the bile juice were measured with an auto-analyzer (model H-747E, Hitachi Co., Tokyo). Lipid peroxide was followed as Masugi and Nagamura stated by thiobarbituric acid method using malondialdehyde (MDA) tetraethyl acetal as the standard and expressed results of MDA equivalents.

Concentrations of malondialdehyde (MDA) in the liver of sham-operated control rats (control group) and rats subjected to 70 min of ischemia and 120 min of reperfusion with (group B) and without (group A) infusion of magnesium ascorbate (100 mg/kg) at 10 min prior to reperfusion process were assayed. Protein contents of the collected tissues were assayed with Lowry using bovine serum albumin as the standard. The collected tissue was fixed in saline buffered with 10% formaldehyde solution, embedded in paraffin wax, sliced into 5 μm thickness of each, stained with H&E staining method, and examined with a light microscope by a pathologist independently.

Statistic Methods

Each data was expressed as mean±SD. Significant comparison between results of each different group was done by Student's t test or Mann-Whitney U test. The survival analysis was done by Wilcoxon signed-rank test.

[Results]

Biochemical Markers of Plasma

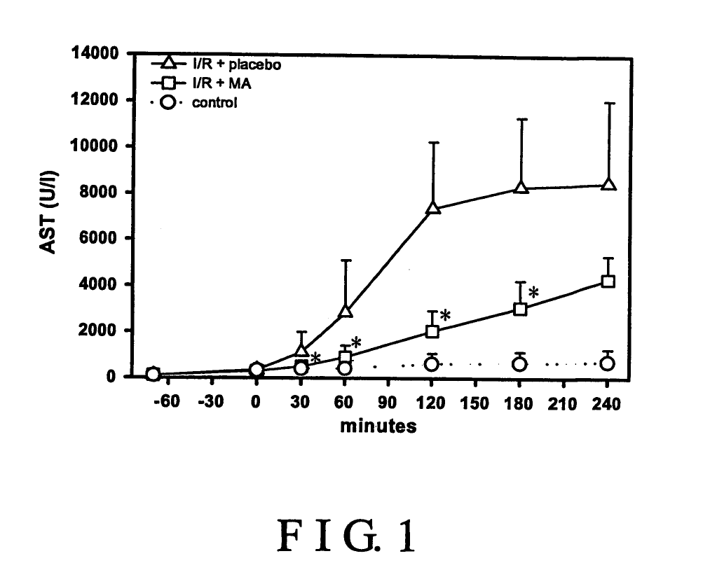

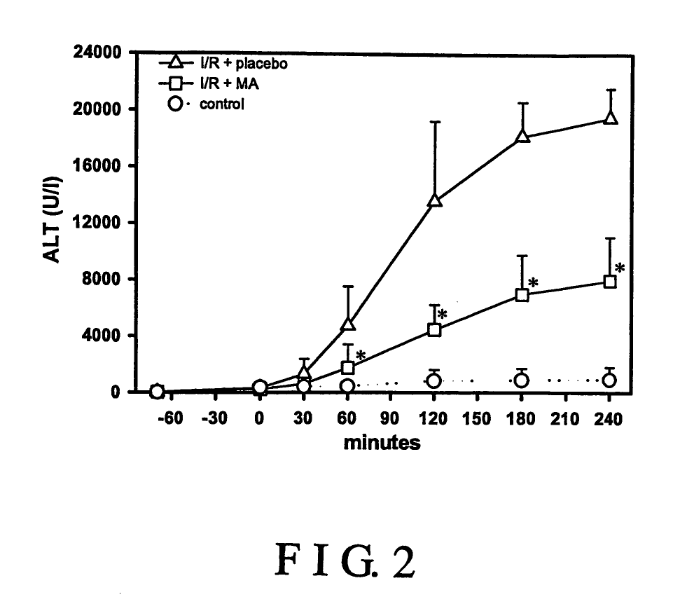

After 70 minutes of ischemia without reperfusion, no significant changes, compared with the pre-ischemic values, were observed in the plasma AST and ALT levels in ischemia rats (FIGS. 1 and 2; standard deviation was marked as *). Compared with the placebo group, plasma AST levels of the MA-treated rats were significantly decreased after 30 minutes of reperfusion (P<0.05). Compared with the placebo group, the plasma AST levels of MA-treated rats were significantly decreased after 60 minutes of reperfusion (P<0.05). However, when the blood flow to the ischemia liver lobes was restored, the average plasma AST level, after 120-minute reperfusion, was 7374±2887 (U/l) in the vehicle-treated ischemia group, and 2040±882 (U/l) in the MA-treated ischemia group, meanwhile the average plasma AST level in the control group was 614±448 (U/l). The plasma ALT level, after 120-minute reperfusion, was 13585±5666 (U/l) in the vehicle-treated ischemia group, and 4466±1745 (U/l) in the MA-treated ischemia group, meanwhile the average plasma ALT level in the control group was 445±367 (U/l). MA significantly decreased 78% of the elevated serum AST level and 69% of the elevated serum ALT level after 120 minutes of reperfusion.

A significant increase was observed in the AST and ALT activity occurring from 1 to 4 hours after reperfusion in the vehicle-treated ischemia rats and significantly suppressed (P<0.05) by the administration of MA (100 mg/kg), as shown in FIGS. 1 and 2.

Bile Juice Analysis

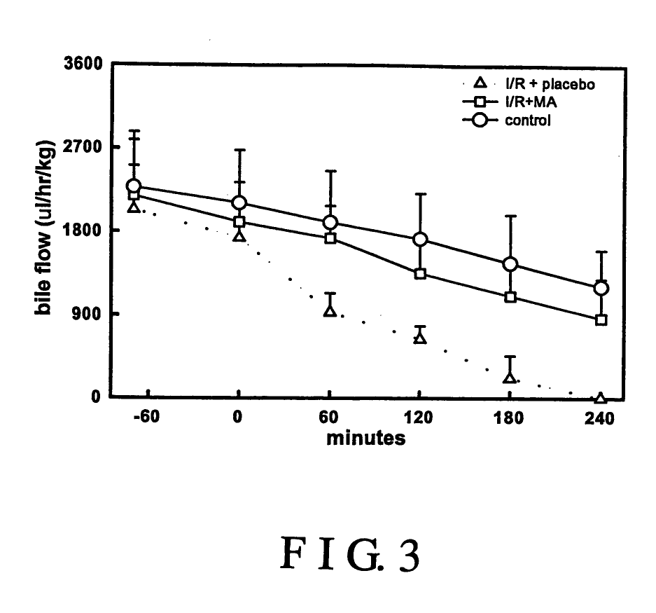

The bile flow was significantly reduced during the ischemia and reperfusion stage, as shown in FIG. 3. The bile flow of MA-treated rats, compared with the control group, was significantly restored resemble to normal values (P=0.21). Choleretic effect of MA on rats, compared with the placebo group, was significantly increased after 60 minutes of reperfusion (P<0.05). Bile flow collected in the vehicle-treated ischemia group after 120 minutes of reperfusion was 639±141 μl/hr/kg, and 1353±351 μl/hr/kg in the MA-treated ischemia group, meanwhile the control group was 1722±489 μl/hr/kg. MA significantly doubled the secreted bile juice after 120 minutes of reperfusion injury. The bile flow, compared with the vehicle-treated ischemia group, was significantly restored in the MA-treated ischemia group from 60 minutes to 240 minutes of reperfusion (P<0.05). The restored bile flow in the MA-treated ischemia group was lower than that in the control group, but the difference was insignificant (P=0.21). Without further treatment, most rats in placebo group secreted only a little bile flow after 240 minutes of reperfusion.

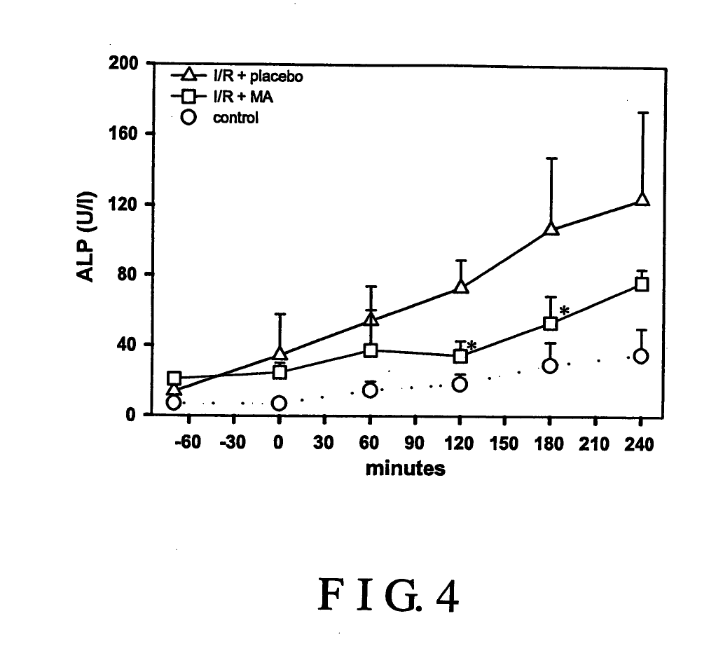

ALP in the bile juice during the I/R process was detected and illustrated in FIG. 4. Compared with the pre-ischemic values, after 70 minutes of ischemia without reperfusion, observed no significant changes in the bile ALP levels of ischemic rats (P>0.05). Nevertheless, bile ALP was observed to elevate significantly after 120 minutes of reperfusion in the vehicle-treated ischemia group (73.3±15.8 (U/l)) and this elevation tendency was significantly decreased by administration of MA (100 mg/kg) to 34.5±8.3 (U/l) after 120 minutes of reperfusion, meanwhile the value in the control group was 18.3±5.8 (U/l). MA could relieve about 70% of the ALP in bile juice from I/R damage. The bile ALP levels of MA-treated rats, compared with the placebo group, were significantly decreased after 120 minutes of reperfusion (P<0.05). Spontaneous increase of bile ALP levels in the sham-operated control group might be attributed to the trauma injured from the surgery.

Lipid Peroxidation Assay

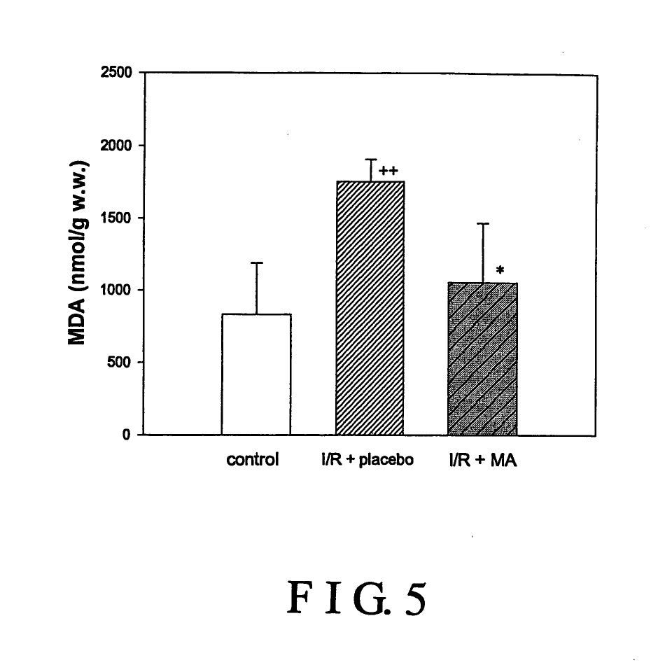

Hepatic I/R resulted in the generation of reactive oxygen species radicals, as shown in MDA levels (FIG. 5). Results were expressed as means±SD for 7-15 rats/group. Compared to the sham-operated control group, MDA content was significantly increased as the result of I/R injury in placebo group (P<0.01; standard deviation was marked as ++). Compared to the placebo group, MA could decrease the MDA content generated from I/R injury (P<0.01). MDA content in MA-treated ischemia group was higher than that in sham-operated control group but it is statistic insignificant (P>0.05). With or without I/R injury, MDA in the vehicle-treated ischemia group (1755±154 (mMol/g)) were significantly higher than those found in control group (833±357 (mMol/g)) (P<0.01). However, those increases in post-ischemic MDA levels were significantly decreased to 1054±414 (mMol/g) when MA (100 mg/kg) was administered (P<0.02).

Survival Rate

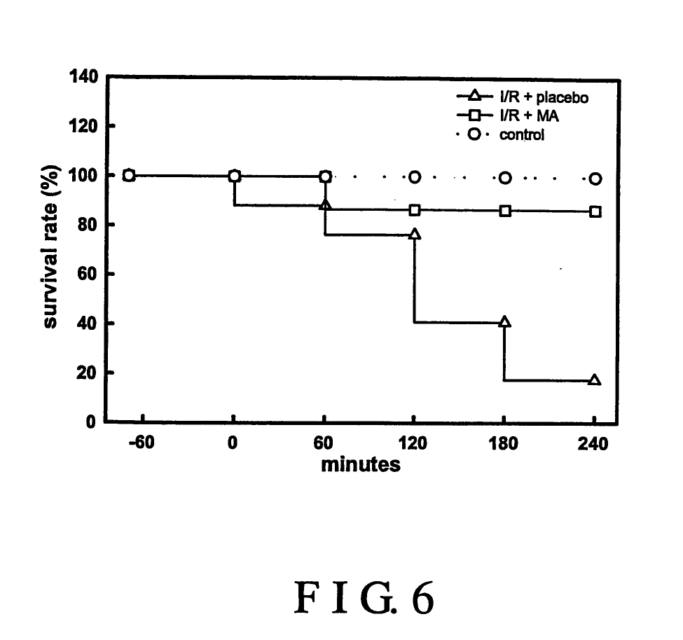

For each of the rats in the experiment, its vital signs were monitored up to 240 minutes of the reperfusion and shown as the recorded survival rate (FIG. 6). The survival rate of MA-treated rats was significantly higher than placebo treated rats after 240 minutes of reperfusion (P<0.05).

Blebbing Test

The blebbing of hepatocytes during the I/R injury experiment was also proven by the loss on dry/wet ration of liver tissue. The liver tissues collected from the experiment were cut into a piece of about 200 mg respectively, weighted, and dried at 80° C. for 48 hours, the dry/wet ratio of the liver was parallel compared.

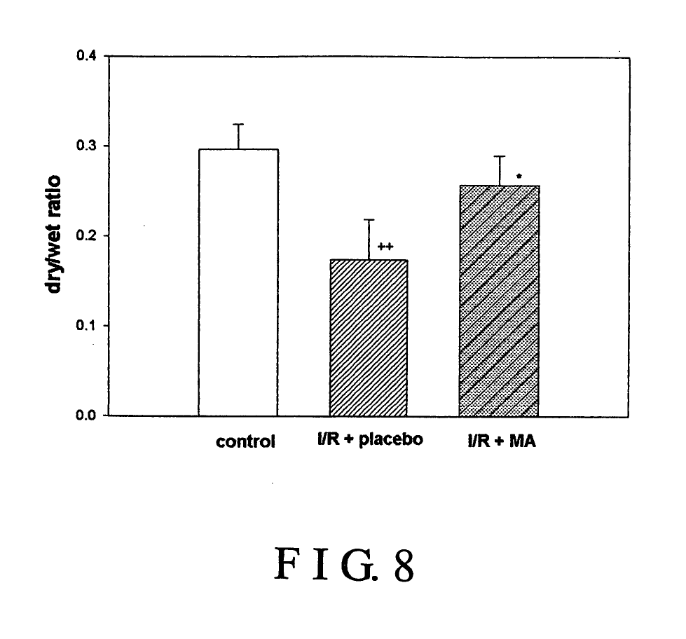

The effect of magnesium ascorbate attenuated the injury of I/R by decreasing the blebbing of heptocytes. Compared with the control group, the vehicle showed no protective effect against the I/R injury and induced hepatocyte swollen (++; P<0.05). On the other hand, the blebbing of hepatocytes was significantly inhibited by the administration of MA (P<0.05). FIG. 8 shows dry/wet ratios of group A, group B and control group in the above experiment.

Histological Investigation of the Liver After Hepatic I/R

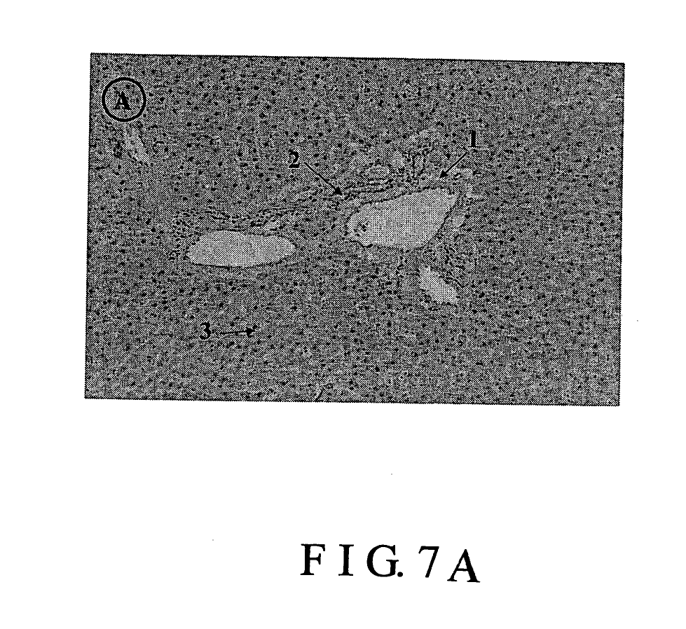

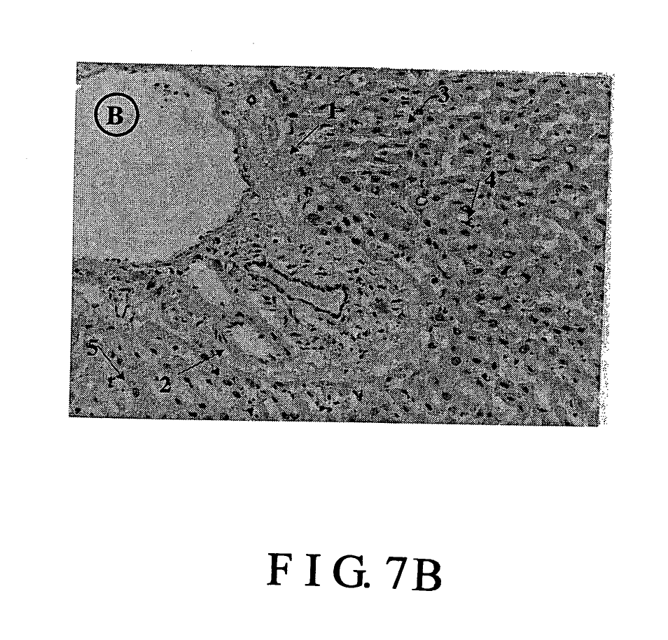

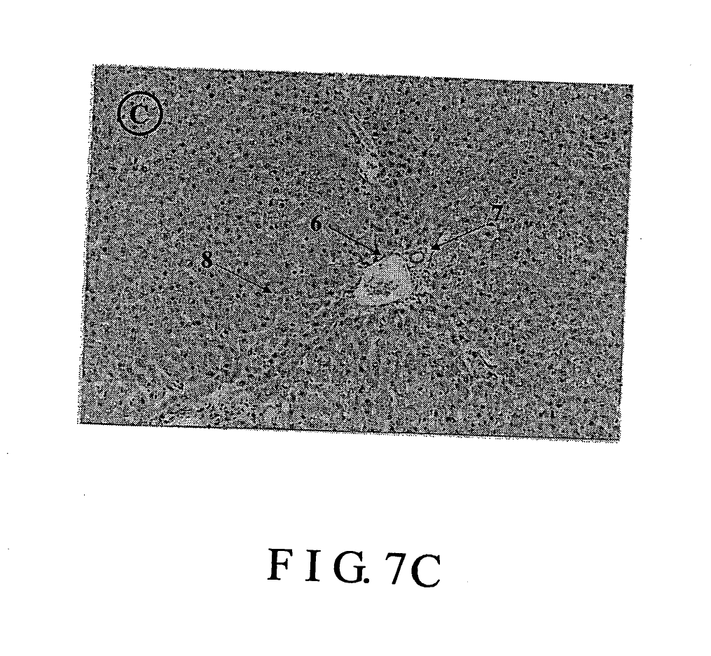

Histological changes in liver after hepatic I/R were kept with the above biochemical experiments of plasma and bile juice analysis. The placebo group was shown on A (100×) and B (400×); wherein “1” is severe endothelial damage, “2” is bile duct dilation, “3,” is parenchyma edema with RBC congestion, “4,” is Cholestasis, and “5” is hepatocyte necrosis. MA-treated rats was showed on C (100×), wherein “6” is a well-protected normal-like endothelium was observed, “7” is a regular size of bile duct, and “8” is parenchyma edema congested with RBC. The dilated bile duct might be attributed to ischemic process when the clamp was used to stop the blood flow and simultaneously obstruct the bile flow. Parenchyma edema and RBC congestion might develop from the inflammatory process during I/R process. It showed that MA could protect most vessels from endothelial injury during blood reperfusion, and prevent cholestasis after I/R process. The histological investigation of the liver in the vehicle-treated ischemia group (FIGS. 7A and 7B) revealed severe congestion of the hepatic areas with multiple and extensive ballooning necrotic hepatocytes randomly distributed among the hepatic parenchyma, severe damage to vein endothelium, and bile stasis of liver. By contrast, in the MA-treated ischemia group (FIG. 7C), minor lesions were observed, damaged to the vascular endothelium and cholestasis was also mild. This parallel pathologic comparison showed that administration of MA could attenuate the injury of hepatic I/R.

Ischemia and reperfusion injury is considered to play a key role in the pathobiology of liver failure after major hepatic resection or transplantation. It is also considered responsible for failure of the primary graft and, in many cases, for triggering a systemic inflammatory response that may lead to multiple organ dysfunction syndromes. The survival rate experiment lends support to these considerations, because, without further treatment, 80% of the animals subjected to 70 minutes of ischemia and 240 minutes of reperfusion died.

MDA is considered as a sensitive index to assess lipid peroxidation generated from reactive oxygen species radicals that exist mostly on damaged cellular membrane. Analytical results of the determination of MDA in liver tissue showed that animals subjected to I/R without prior administration of MA contained significantly higher MDA concentration than the control animals (FIG. 5). These results provide additional evidence that I/R triggers lipid peroxidation. The above hepatic pathology observed severe damage to veins endothelium, hepatocyte necrosis of portal area and parenchyma congestion syndrome could account for this reactive oxygen species damage generated from Fenton reaction during the reperfusion.

Pretreatment at 10 minutes prior to reperfusion by MA (100 mg/kg) infusion significantly decreased the MDA concentration in liver tissue. Higher mean MDA level in the vehicle-treated ischemia group was alleviated in the MA-treated ischemia group after 120 minutes of reperfusion (see FIG. 5). It showed that MA could significantly attenuate the MDA generated during I/R process. Such effect might be attributed to the ascorbate antioxidant in the MA. The validity of MDA assay as an index of lipid peroxidation in biological material somehow had been clouded by controversy artifact analysis during the test itself, its occurrence in various bound forms, and the specific of the techniques used for its determination was discussed. But there are theoretical objections to other alternative assays now available for investigating free radical production in clinical practice, and the MDA assay has the merit of simplicity.

AST and ALT were well known as biochemical markers index for inflammation, and such inflammatory effect was known related with the reoxygenation process during the I/R process. A significant increased in the AST and ALT values, compared with the sham-operated control group, was exhibited by the animals subjected to I/R injury with or without pre-treatment (as showed in FIGS. 1 and 2). Such corroborated the inflammatory effect generated during the I/R process. This plasma AST and ALT elevation well correlated with the pathological observations of hepatic parenchyma congestion and hepatocyte necrosis (FIGS. 7A and 7B). Seo took this parallel correlation as association exit between increased lipid peroxidation and hepatocyte injury. In the present invention, infusion of MA would alleviate these inflammatory parameters generated from I/R injury after 120 minutes of reperfusion (see also FIGS. 1 and 2).

It is well known that bile flow is a very important indicator for liver functions. In this experiment, the bile flow of the ischemia liver lobes was obstructed and a significant decrease was observed after 60 minutes of the reperfusion on vehicle-treated ischemia group (P<0.05) (see FIG. 3). Such results well correlated with previous reports that the major injury of I/R developed on the reperfusion stage. In the MA-treated ischemia group, although the mean bile flow was lower than that in the control group, it is statistically insignificant (P>0.05). Such choleretic effect of MA was proven through pathological examination of liver tissue, and there was comparably much less cholestasis observed in the MA-treated ischemia group than the vehicle-treated ischemia group (see FIGS. 7B and 7C). It is known that bile is secreted from hepatocytes and increases parallel with healthy hepatocyte number. The choleretic effect of MA may significantly make the magnesium to preserve glucose and pyruvate levels to preserve cellular energy metabolism. Such energy preservation made less damage to hepatocytes due to energy depletion during the ischemic process. The level of bile ALP that rose in cholestasis in the vehicle-treated ischemia group was relieved by MA treatment in the MA-treated ischemia group, which would also reinforce this observed choleretic effect of MA (see FIG. 4). Collective effects of ascorbate and magnesium seemed to tremendously contribute to combating I/R injury and increase the survival rate significantly (shown in FIG. 6).

In conclusion, magnesium ascorbate (MA) has both ascorbate antioxidant and magnesium energy preservation properties to attenuate ischemia/reperfusion injury, and therefore significantly increase the survival rate after liver ischemia/reperfusion injury on rats (P=0.0002).

While the above examples illustrate the preferred embodiment of the present invention, from the examples it may be easily deduced that the protective effects of magnesium ascorbate (MA) should not be limited in liver ischemia/reperfusion injury, but all types of ischemia/reperfusion injury.

What is claimed is:

1. A pharmaceutical composition for treating and/or preventing ischemia/reperfusion injure, comprising magnesium ascorbate as an active ingredient; and a pharmaceutically acceptable carrier.

2. The pharmaceutical composition as claimed in claim 1, which is used for treating and/or preventing liver ischemia/reperfusion injure.

3. The pharmaceutical composition as claimed in claim 1, wherein the magnesium ascorbate is administered with 1˜500 mg/kg of body weight.

4. The pharmaceutical composition as claimed in claim 3, wherein 1.0 g of magnesium ascorbate comprises 600˜930 mg of Vitamin C and 2˜350 mg of magnesium.

5. The pharmaceutical composition as claimed in claim 3, wherein 1.0 g of magnesium ascorbate comprises 800˜900 mg of Vitamin C and 6˜100 mg of magnesium.

6. The pharmaceutical composition as claimed in claim 1, wherein the magnesium ascorbate is administered with 75˜125 mg/kg of body weight.

7. The pharmaceutical composition as claimed in claim 6, wherein 11.0 g of magnesium ascorbate comprises 600˜930 mg of Vitamin C and 2˜350 mg of magnesium.

8. The pharmaceutical composition as claimed in claim 6, wherein 1.0 g of magnesium ascorbate comprises 800˜900 mg of Vitamin C and 6˜100 mg of magnesium.

9. A method for treating and/or preventing ischemia/reperfusion injure in mammals, comprising administering a therapeutically effective amount of magnesium ascorbate to a mammal.

10. The method as claimed in claim 9, which is used for treating and/or preventing liver ischemia/reperfusion injure.

11. The method as claimed in claim 9, wherein the therapeutically effective amount of magnesium ascorbate is 75˜125 mg/kg of body weight.

12. The method as claimed in claim 11, wherein 1.0 g of magnesium ascorbate comprises 600˜930 mg of Vitamin C and 2˜35 mg of magnesium.

13. The method as claimed in claim 11, wherein 11.0 g of magnesium ascorbate comprises 800˜900 mg of Vitamin C and 6˜10 mg of magnesium.

14. The method as claimed in claim 9, wherein therapeutically effective amount of magnesium ascorbate is 1˜500 mg/kg of body weight.

15. The method as claimed in claim 14, wherein 1.0 g of magnesium ascorbate comprises 600˜930 mg of Vitamin C and 2˜350 mg of magnesium.

16. The method as claimed in claim 14, wherein 11.0 g of magnesium ascorbate comprises 800˜900 mg of Vitamin C and 6˜100 mg of magnesium.
